(19) United States
(12) Patent Application Publication
(10) Pub. No.: US 2020/0375500 A1
(43) Pub. Date: Dec. 3, 2020

(54) SYSTEMS AND METHODS FOR DETECTION AND CORRECTION OF ABNORMAL MOVEMENTS

(71) Applicant: LOMA LINDA UNIVERSITY, Loma Linda, CA (US)

(72) Inventors: Salvador SORIANO, Loma Linda, CA (US); Michael CASTELLO, Loma Linda, CA (US)

(21) Appl. No.: 16/970,686

(22) PCT Filed: Feb. 22, 2019
(86) PCT No.: PCT/US2019/019149

§ 371 Date: Aug. 18, 2020

Related U.S. Application Data
(60) Provisional application No. 62/634,474, filed on Feb. 23, 2018.

(51) Int. Cl. A61B 5/11; A61B 5/0488; A61B 5/00

(52) U.S. Cl. CPC A61B 5/1101; A61B 5/0488; A61B 5/6824; A61B 5/0022; A61B 2562/0219; A61B 5/4082; A61B 5/4088; A61B 5/721; A61B 5/7475; A61B 5/002

(57) ABSTRACT

Systems and methods for identifying abnormal movements or tremors in one or more human subjects. Kinetic and/or electromyographic sensors are employed in detection hardware to detect voluntary and involuntary movements. The data collected from such voluntary and involuntary movement detection can be further processed and compared to baseline data to identify and distinguish abnormal movements from normal movements. The identification of abnormal movements may indicate a neurodegenerative disorder.

### RELATED APPLICATIONS

This application is a PCT application, and claims priority to, and the benefit of, U.S. Provisional Application No. 62/634,474, filed Feb. 23, 2018, titled “SYSTEMS AND METHODS FOR DETECTION AND CORRECTION OF ABNORMAL MOVEMENTS,” which is incorporated herein by reference in its entirety.

### BACKGROUND

### 1. Field of Invention

The present technology relates to detection of abnormal movements in human subjects, and more particularly to detection, measurement, and monitoring of abnormal movements such as tremors, myoclonus, chorea, athetosis, balismus, bradykinesia, and more.

### 2. Description of the Prior Art

Movement disorders, such as, for example, essential tremor and Parkinson's disease, can have a significant adverse effect on quality of life. Such disorders can diminish the ability to carry out everyday tasks, such as eating, dressing, writing, and using a computer. Such disorders can also impact social interaction and function. In addition, many movement disorders can be difficult to distinguish from one another, principally due to the fact that diagnosis relies on the use of subjective, non-quantitative scales and tests carried out at discrete times. These momentary snapshots of movement patterns in a subject are poorly suited for mechanistic studies aiming to address the etiology of a condition, and cannot reliably monitor changes during periods of time between clinic visits.

Moreover, many people who suffer from neurological conditions do not visit a neurologist until after neurodegeneration has occurred, at which time many therapies have a decreased effectiveness. Thus, additional tools are needed to increase detection accuracy, to allow continuous monitoring or relevant parameters between visits to the clinic, and to detect pre-symptomatic stages of tremor conditions.

### SUMMARY

In one or more embodiments, a device detects tremors or abnormal movements in a human subject or patient. The detection of abnormal movements can be a symptom of a neurodegenerative disorder, such as Parkinson's disease. The device is arranged and designed to use one or more sensors, which may be attached to or positioned in close proximity to the human subject. The sensors can include one or more of an accelerometer to measure muscle movement in the human subject and generate muscle movement data, an electromyographic sensor to measure electrical impulses generated by muscles of the human subject and generate electrical impulse data, and a gyroscope to measure muscle movement in the human subject and generate muscle movement data. In some embodiments, signal processing, e.g., through computer-implemented, executable instructions or software, can be applied to data generated by the motion-sensing device to identify abnormal movements. The muscle movement data and electrical impulse data are compared to predetermined baseline data to identify abnormal movements of the human subject.

In one or more embodiments, a method is disclosed for measuring tremor activity or abnormal movements in a human subject or patient. The method may include detecting muscle movement in the human subject with a kinetic sensor, detecting electrical impulses from muscles in the human subject with an electromyographic sensor, comparing data associated with the muscle movement and the electrical impulses with predetermined baseline data to identify abnormalities in the data, and analyzing the abnormalities in the data associated with the muscle movement and the electrical impulses to identify abnormal movement in the human subject. In some embodiments, signal processing can be applied to the data collected from the various sensors of the motion-sensing device or detection hardware to identify abnormal movements.

In one or more embodiments, a computer-implemented method, under the control of one or more computer systems configured with executable instructions, detects both intended motion and involuntary motion of a human subject or user with an input sensor, generates a distorted instruction signal including data related to both the intended motion and the involuntary motion, sends the distorted instruction signal to a signal filter, produces a corrected instruction signal stripped of the involuntary motion, and sends the corrected instruction signal to a device driver. Thus, in some embodiments, abnormal movement signals identified through the method can be used to eliminate abnormal signals from motion-based computer input.

Detection devices and signal processing computer programs or software, as disclosed herein, can identify individuals with early signs of movement disorders. Such technology has the potential to substantially improve the quality of life of a human subject suffering from a neurodegenerative disorder, such as Parkinson's disease.

### BRIEF DESCRIPTION OF THE DRAWINGS

FIG. 1 shows a first flow chart of an example embodiment of a method for identifying the presence of abnormal movement in a person.

FIG. 2 shows a second flow chart of an example embodiment of a method for identifying the presence of abnormal movement in a person.

FIG. 3 shows a schematic block diagram example embodiment of an abnormal movement detection system.

FIG. 4 shows a schematic block diagram example embodiment where an instruction signal from a motion input device can pass through an abnormal movement filter to remove interfering abnormal movement signal before being interpreted by a device driver.

FIG. 5 shows a flow chart of an example embodiment for calibrating methods of detecting abnormal movement.

FIG. 6 shows a screen shot of a random sequence of color change used in an experimental task performed pursuant to an embodiment of the present technology.

FIG. 7 shows a scatter plot classifying data collected during experiments related to an embodiment of the present technology.

FIG. 8 shows a frequency vs. time plot and associated spectral representation of data collected from an accelerometer.

FIG. 9 shows a frequency vs. time plot and associated spectral representation of data collected from a gyroscope.

FIG. 10 shows a frequency vs. time plot and associated spectral representation of data collected from an electromyographic sensor.

FIG. 11 shows a flow chart of an example experimental procedure according to an embodiment of the present technology.

### DETAILED DESCRIPTION OF CERTAIN EMBODIMENTS

Abnormal movement can comprise tremors, myoclonus, chorea, athetosis, balismus, bradykinesia, or other involuntary movements associated with or indicative of a potential neurological problem. Identifying an abnormal movement in patients and differentiating between types of abnormal movement sometimes requires neurological evaluation by a trained neurologist. Unfortunately, many people do not visit a neurologist until significant neurodegeneration has occurred and therapies are less effective. Thus, automated approaches that can be performed with commercially available devices are beneficial as they can identify those people who are at risk and direct them to seek out medical care.

Automated approaches of sufficient sensitivity may also be used to assist in abnormal movement diagnosis by a neurologist. Automated approaches for detection of abnormal movement (e.g., such as Parkinson's disease, the most common form of tremor in the elderly) can include vocal analysis and the use of accelerometers, such as those found on modern smartphones. Movement can be detected by using a kinetic sensor, such as an accelerometer, possibly with the additional use of a gyroscope. Use of accelerometer data for detecting abnormal movement (such as via a smartphone) relies on detecting movement abnormalities, which may already be hindering a person's ability to function normally, and can be confounded by fatigue or the surrounding environment. Methods of movement detection using accelerometers are known as accelerometry.

Electrical impulses can be measured to detect muscle movements indicating abnormal movement. Muscle activity can create electrical impulses, which may be measured to detect muscle movement even if no movement is detected visually or by an accelerometer. Electrical impulses can also be detected to identify physical actions such as arm rotation or forming a fist. By measuring electrical impulse data in addition to movement data, sensitivity and accuracy of abnormal movement detection can be greatly increased and can take place at an earlier time in the disease process when the individual may be less impaired. With sufficiently sensitive detection hardware, abnormal movement may be detected even when visual diagnosis of abnormal movement may not be possible. One method of movement detection based on the measurement of electrical impulses is known as electromyography (EMG).

Abnormal movements can be detected by comparing a detected electrical impulse signal and movement signal to identify discrepancies or inconsistencies. Abnormal movements can also be detected by evaluating a motion signal built from both movement data (such as from an accelerometer) and an electrical impulse signal. Using both movement data and muscular electrical impulse data for abnormal movement detection improves the accuracy and sensitivity of abnormal movement detection. When used separately, both accelerometry and EMG provide only a partial picture of a particular tremor phenotype. In addition, since both methods are used to test abnormal movement after patients have already reached a stage where tremors affect daily function, the potential for either method is limited when applied in isolation.

According to embodiments of the present technology, accelerometry and EMG can be used simultaneously to measure kinematic and electrical impulse patterns in order to produce reproducible, objective, and quantitative results. By combining data from both accelerometry and EMG evaluation, sensitivity and accuracy of tremor detection can be greatly increased, to levels that allow a neurologist or other clinician to reproducibly, objectively, and quantitatively identify early stages of a disorder. The advantages of the present technology are many, including early intervention in treatment for particular disorders, leading to better treatment outcomes, to provide independence to patients that are unable or unwilling to visit a clinician regularly, and to find and compensate for specific tremor patterns, potentially allowing impaired people to complete tasks such as operating a computer, where they otherwise could not. In addition, early detection can help to further the study and understanding of movement disorders by helping in the design of patient allocation criteria in clinical trials.

Hardware or a device for detecting movement and electrical impulses can be worn on the arm or another body portion of a user. The detection hardware can in some embodiments be worn as a bracelet. Detection hardware can be capable of sensing three-dimensional movement as well as detecting small electrical impulses generated by muscle movement. A gyroscope and accelerometer can be used to track three-dimensional movement. An electromyographic sensor, e.g., along with electrode leads attached to a user, can detect small electrical impulses generated by muscle movement. The detection hardware can in some embodiments include one or more of an accelerometer, an electromyographic sensor, electrode leads, and a means of communicating data with a computer. Other sensors for detecting movement or detecting electrical impulses generated by muscle movement can also be used. Data can be communicated from the detection hardware with a computer in many ways including through a wired connection, through a wireless local area network, through a Bluetooth connection, or through a cellular telephone link. In some embodiments the detection hardware can comprise of the MYO device developed by Thalmic Labs, Inc. of Kitchener, Ontario, Canada.

Based on movement and electrical impulse data, early signs of abnormal movement can be detected. Signal isolation algorithms can be used to identify some patterns specific to certain pathologies such as Parkinsonian tremor or other varieties of abnormal movement. Software features of some embodiments can include any combination of means of acquiring movement and/or electrical impulse data from hardware, processing that data to identify abnormal movements, and filtering an abnormal movement signal from the final data stream before sending it to a computer.

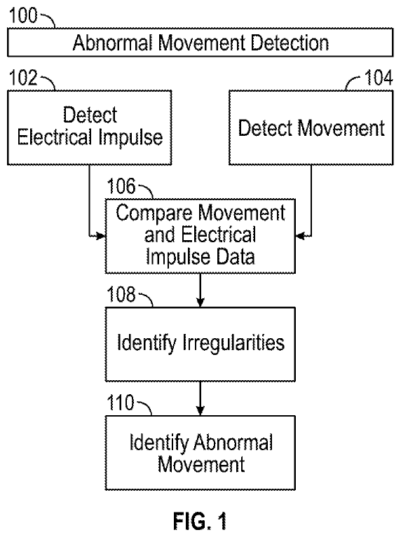

FIG. 1 shows a flow chart example of a method for detecting abnormal movements 100 utilized in some embodiments of the present disclosure. First, both electrical impulse data is detected 102, and movement data is detected 104. Detection 102 and 104 can include identifying the presence of movement or electrical impulses, and in some embodiments can include measuring any of the direction, magnitude, frequency, or duration of movement or electrical impulses. In some embodiments the detection of the movement data 104 and the electrical data 102 can be accomplished by a single article of detection hardware capable of detecting both movement and electrical impulses. In some embodiments the detection of the movement data 104 can be accomplished by a separate article of detection hardware from the detection of electrical impulses 102.

Movement data and electrical impulse data for detection 104 and 102 can be detected in a variety of settings. Detection hardware worn by a user can passively detect movement and electrical impulse data during the course of normal daily user activity. In some embodiments movement data and electrical impulse data can be detected during supervised user activity specifically for the purpose of evaluating potential abnormal movements. Movement instructions can be given to a user to facilitate abnormal movement detection. A neurologist or other medical professional can observe or direct the movements of a user wearing detection hardware. A user can wear detection hardware in accordance with a medical professional's instruction between visits to the medical professional in order to provide data to the medical professional for the purpose of diagnosing abnormal movement. In some embodiments a user can provide input regarding motion to be performed while wearing the detection hardware.

Second, detected electrical impulse data and detected movement data from the same time frame can be compared 106. Detected electrical impulses from muscle movements can be correlated to specific detected movement. Correlated data can be compared. Electrical impulses from muscles can be detected shortly before any movement. Some muscle movements can produce known or predictable electrical impulses. For comparison with detected data 106, in some embodiments, specific muscle activity and associated electrical impulses can be predicted based on a detected movement. Predications of electrical impulse data can in some embodiments be made based on previously collected movement and electrical impulse data from the same user or other users. Predictions of electrical impulse data can also be based on any known properties of the detected movement, such as what movement (if any) was intended by the user at the time of the data detection.

Third, irregularities or inconsistencies in the detected data can be identified 108. In some embodiments instances can be identified where the detected electrical signal may not be as expected based on the detected movement. Deviations in the detected electrical impulses from the expected or predicted electrical impulse for a detected movement can be measured. Aberrations in the detected electrical impulse data preceding movement can also be detected. What qualifies as an irregularity or inconsistency for identification 108 can in some embodiments be determined differently for an individual user. Previously collected data from users both exhibiting abnormal movement and those not exhibiting abnormal movement can be considered to determine if detected data reflects an irregularity or inconsistency. The direction, magnitude, frequency, or duration of any irregularity in the detected data can also be identified 108. In some embodiments signal isolation algorithms can be applied to the detected data in order to identify potential abnormal movements (such as, for example, Parkinsonian tremor).

Fourth, based on any identified irregularities or inconsistencies, an abnormal movement or potential abnormal movement can be identified 110. In some embodiments the identification of abnormal movement or abnormal movement pattern 110 can be based on variations in detected electrical impulse data from the electrical impulses expected with a detected movement. Where detected electrical impulses indicate unusual muscle activity for a detected movement, an abnormal movement could potentially be present. If detected electrical impulses would normally be associated with a movement that was not detected, the impulses could indicate abnormal movement or a potential abnormal movement not yet detectable by accelerometer or by visual inspection. Identification of abnormal movement 110 can include identification of a potential abnormal movement, the probability of abnormal movement, or the need to follow up with a neurologist. Where abnormal movement is not indicated by any irregularities or inconsistencies, identifying abnormal movement 110 can include identifying an absence of abnormal movement.

Differences between abnormal movements can be identified based on the detected electrical impulse and movement data. An abnormal movement can be characterized by identifying the magnitude, duration, or frequency of movements detected correlating to an irregularity in the electrical impulse data. An abnormal movement can also be characterized by identifying the magnitude, duration, or frequency of an identified irregularity in the electrical impulse data. In some embodiments abnormal movements can be differentiated based on the context that the data indicating an abnormal movement was detected. For example, if an abnormal movement is indicated when a user wearing the detecting hardware is at rest, then the abnormal movement may potentially be characterized as a resting tremor. If an abnormal movement is indicated when a user is making some volitional movement, then the abnormal movement may potentially be characterized as an intention tremor. Other differentiations may be made between identified abnormal movements based on known context for an identified abnormal movement and any determinable features of an identified abnormal movement. For example, an abnormal movement can in some embodiments be identified as any of tremor, myoclonus, chorea, athetosis, balismus, bradykinesia, or other involuntary movements associated with or indicative of a potential neurological problem.

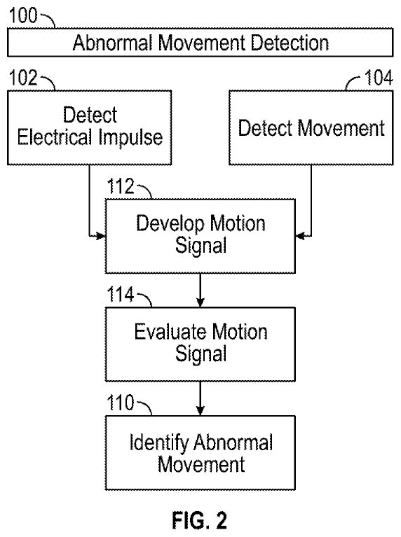

FIG. 2 shows a different flow chart example of the method for detecting abnormal movements 100 utilized in some embodiments of the present disclosure. First, both electrical impulse data is detected 102, and movement data is detected 104. The detection of data 102 and 104 for the method of FIG. 2 can be accomplished in any manner described above for FIG. 1. Detection of data 102 and 104 can include measuring any of the direction, magnitude, frequency, or duration for movement or electrical impulses. As described above in regards to the method of FIG. 1, the detection of data 102 and 104 can be accomplished in some embodiments by a single article of detection hardware, or multiple articles of detection hardware. Similarly, movement data and electrical impulse data for abnormal movement detection can be detected in a variety of settings, including the settings and embodiments described above for FIG. 1.

Second, a motion signal can be developed based on the detected data 112. A motion signal can reflect the resulting motion associated with both the detected movement and the detected electrical impulses from muscle activity. In some embodiments motion signal development 112 can consist of identifying electrical impulse data and movement data. In some embodiments the motion signal can be developed 112 by adjusting detected movement by applying motion corrections for detected electrical impulses indicating muscle activity. For example if a small muscle action is detected via electrical impulses, a small motion may be inferred, and, if not already reflected in the detected movement, the inferred motion could be used to adjust and develop the motion signal 112. The amount of motion to infer from a detected electrical impulse can be determined in some embodiments based on previously collected data from the same user, similar users, or other users. By including indications of muscle movement from detected electrical impulses, the motion signal development 112 can in some embodiments include both motion large enough to be detected visually and motion not large enough to be detected visually.

Third, the developed motion signal can be evaluated 114. Evaluation of a motion signal 114 can include isolating or locating patterns within a motion signal. Small or large irregularities in a motion signal can be identified. Delays or interruptions in the progression of a motion can also be identified in the signal. In some embodiments the direction, magnitude, frequency, and duration of patterns in a motion signal can be evaluated. The evaluation of the motion signal 114 can include comparisons to previously collected data or other developed motion signals from different sets of data. In some embodiments a motion signal can be compared to a previously developed motion signal known to indicate the presence of abnormal movement. Motion signal evaluation 112 can also include consideration of any known context for the detected data such as the known intended motions of a user while wearing the detection hardware. In some embodiments signal isolation algorithms can be applied to the motion signal to locate potential abnormal movements (such as, for example, potential Parkinsonian tremor).

Fourth, based on the evaluation of the developed motion signal, the presence or absence of abnormal movement can be identified 110. In some embodiments abnormal movement identification 110 can include comparing patterns found in the motion signal to patterns found in previously developed motion signals known to be associated with abnormal movement. In some embodiments abnormal movement identification 110 can include detecting irregularities, delays, or interruptions in motion. Features such as magnitude, frequency, and duration of patterns in a motion signal can be used to identify abnormal movement 110. The criteria applied to an evaluated motion signal to identify abnormal movement can 110 be specific to an individual user or based on previously known features of motion signals associated with abnormal movement. In some embodiments a neurologist or physician can use a developed motion signal to identify an abnormal movement, with or without using other diagnostic tools.

As described with regards to the identification of abnormal movement in the method of FIG. 1, the identification of abnormal movement 110 can include differentiating between types of abnormal movement. For example, an abnormal movement can in some embodiments be identified as any of tremor, myoclonus, chorea, athetosis, balismus, bradykinesia, or other involuntary movements associated with or indicative of a potential neurological problem. Features of the motion signal including the frequency, magnitude, or duration of any patterns in the signal can indicate characteristics of an identified abnormal movement. Any known context for user motion associated with a motion signal can be used to characterize an identified abnormal movement. Identification of abnormal movement 110 can also include identification of the probability that abnormal movement is present, or the need to follow up with a neurologist on potential abnormal movement. As described above, the identification of abnormal movement 110 can include the identification of the absence of abnormal movement.

While described in separate flow charts, in some embodiments multiple methods for detecting abnormal movement 100 can be utilized. A single set of detected electrical impulse and movement data can be used both for comparing data 106 in the method of FIG. 1, and used for developing a motion signal 112 in the method described in FIG. 2. Abnormal movement detection can be accomplished through a combination of the elements described in relation to the methods of FIG. 1 and FIG. 2. In some embodiments an abnormal movement can be identified and characterized both by deviations of the electrical impulse data from the expected electrical impulse data for a detected movement, and by evaluating features of a developed motion signal. The same detection hardware and processing means can be used to perform both the method of FIG. 1 and of FIG. 2.

Comparisons of data 106, development of a motion signal 112, identification of irregularities or inconsistencies 108, evaluation of a motion signal 114, identification of abnormal movement 110, and any other manipulation of the detected data can in some embodiments be performed on one or more processors. In some embodiments a hardware processor can be used to perform one or more manipulations of the detected data. Data from the detection hardware can be communicated to a personal or hand-held computer including at least one processor. In some embodiments some manipulations of the detected data can occur in one or more processors within the detection hardware. While described in separate steps of the flow charts, each step can be performed on the same processor, or in some embodiments, multiple processors can be used.

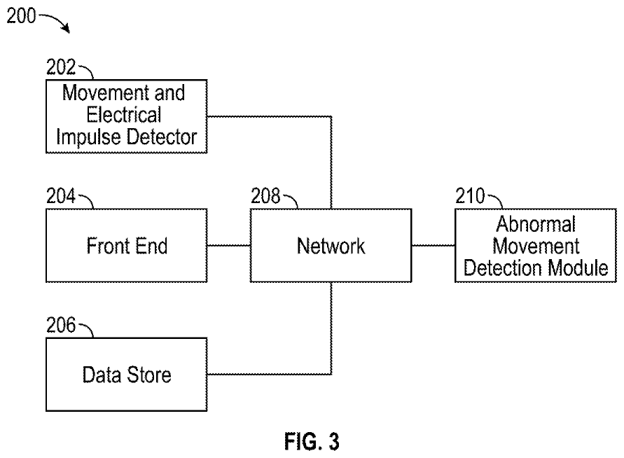

FIG. 3 shows a schematic example of an embodiment of an abnormal movement detection system 200. The abnormal movement detection system 200 can apply abnormal movement detection module 210 to data detected by movement and electrical impulse detector 202. Any abnormal movements detected or identified by the abnormal movement detection module 210 can in some embodiments be presented to a user through the front end 204. The data store 206 can maintain a record of user data, data regarding identified abnormal movement patterns, abnormal movement detection data from abnormal movement detection module 210, detected movement data or electrical impulse data from movement and electrical impulse detector 202, measurements of detected data or abnormal movement, relationships between sets of data, other medical data, or a combination of different types of data.

The movement and electric impulse detector 202 can be any detection hardware capable of both detecting electrical impulses generated by muscle activity, and detecting movement. Movement and electrical impulse detector 202 can comprise one article of detection hardware or multiple articles of detection hardware. The movement and electrical impulse detector 202 can comprise of a single article of detection hardware. In some embodiments movement and electrical impulse detector 202 can comprise of multiple articles of detection hardware. Although not illustrated, in some embodiments vocal analysis for abnormal movement can also be incorporated in abnormal movement detection system 200 to provide additional abnormal movement detection capabilities.

An abnormal movement detection module 210 can apply a variety of methods to identify an abnormal movement based on the detected data. Electrical impulse data and movement data detected by the electrical impulse and movement detector 202 can be evaluated or compared by abnormal movement detection module 210 to detect abnormal movement. The abnormal movement detection module 210 can apply the method for detecting abnormal movement described in FIG. 1. In some embodiments the abnormal movement detection module 210 can apply the method for identifying abnormal movement described in FIG. 2. The abnormal movement detection module 210 can also apply multiple methods for identifying abnormal movement, including any combination of the elements of the methods described in FIG. 1 and FIG. 2. The abnormal movement detection module 210 can include one or more processors. In some embodiments data recorded in data store 206, such as previously identified abnormal movement patterns, can also be used by abnormal movement detection module 210 to identify abnormal movement.

Once abnormal movement, or the absence of abnormal movement, is identified by the abnormal movement detection module 210, a user can in some embodiments access information about any detected abnormal movement through the front end 204. The front end 204 can provide information including (among other possibilities): the probability an abnormal movement was detected, the correlated movement data and electrical signal data associated with an identified abnormal movement, any possible characterizations of an identified abnormal movement, or an indicated absence of abnormal movement. The abnormal movement detection module 210 can in some embodiments provide the front end 204 with depictions of the data relied on for identification of abnormal movement. As described above, data relied on for identification of abnormal movement can include (among other possibilities) detected movement and electrical impulse data, expected electrical impulse data associated with a detected movement, previously collected electrical impulse data for movement data known to be associated with abnormal movement, or a previously evaluated motion signal.

The front end 204 can in some embodiments include a display module. The display module can display information related to both the detected data and any identified abnormal movements or potential abnormal movements. A display module can include one or more screens for displaying data. A display module can in some embodiments be part of a computer display, a tablet display, or a smartphone display. In some embodiments front end 204 can also include audio features to provide information to a user or neurologist. Audio features can include alerts for the detection of abnormal movement or detection of abnormal movement meeting user specified criteria.

In some embodiments a user can interact with the front end 204 to select what results or correlating data is displayed. The level of detail displayed regarding the abnormal movement identification process can be adjusted for different users. If, as in some embodiments, the abnormal movement detection system 200 is being used for personal evaluation of the likelihood of abnormal movement, then the front end 204 can provide a calculation of the percent chance of abnormal movement detection along with a recommendation of whether to follow up with a neurologist. If the abnormal movement detection system 200 is being used to assist a neurologist or other medical professional in diagnosing an abnormal movement, more detail can be displayed. A user can in some embodiments interact with the front end 204 to alter the focus, signal processing, or time portion displayed for any data or results being displayed.

The movement and electrical impulse detector 202, the front end 204, the data store 206, and the abnormal movement detection module can connect to one another through a network 208. The network 208 can include a local area network, a wide area network, a wired network, a wireless network, a local bus, a Bluetooth connection, connection to a cellular phone network, or any combination thereof. In some embodiments, one or more components of the system 200 connect another component of the system 200 over the Internet. While movement and electrical impulse detector 202, front end 204, data store 206 and abnormal movement detection module 210 are pictured as separate modules, in some embodiments features of each module can overlap. In some embodiments one or more processors may be utilized by one or more of movement and electrical impulse detector 202, front end 204, data store 206 and abnormal movement detection module 210.

When used by a neurologist or other medical professional, some embodiments of the disclosure may allow for a more accurate and detailed understanding of a patient's abnormal movement. As described above, the methods for detecting abnormal movement using both electrical impulse and movement detection can provide a greater degree of sensitivity in identifying signs of abnormal movement because small motions of muscles indicated only by electrical impulses may not be detectable by visual inspection. In some embodiments where a patient wears detection hardware for passive electrical impulse and movement detection between visits to a neurologist, the collected data may allow for a more accurate track of the progression of abnormal movement symptoms. Features of a patient's identified abnormal movements or potential abnormal movements can also be tracked by a neurologist to help determine how abnormal movement symptoms are progressing.

In some embodiments the features of an identified abnormal movement or potential abnormal movement can be evaluated to determine the risk of mild cognitive impairment (MCI) progressing to Parkinson's disease or to Alzheimer's disease. While abnormal movement and MCI can both be indicative of a risk to progress to Parkinson's disease or Alzheimer's disease, the abnormal movements exhibited may be used to distinguish whether Parkinson's or Alzheimer's is likely to develop. By tracking the features of a patient's abnormal movements with some embodiments of the present disclosure, a neurologist can assess the risk that MCI will progress to Alzheimer's. Enabling early differentiation between patients with MCI and abnormal movement can increase treatment options or efficacy and has the potential to improve a patient's prognosis.

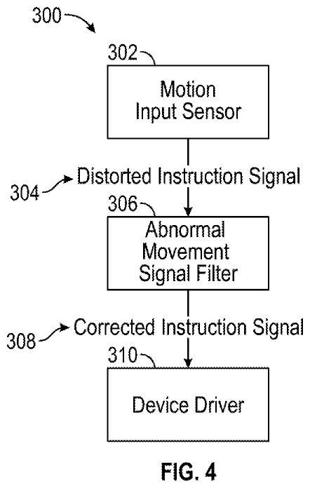

In some embodiments the abnormal movement correction system can compensate for specific abnormal movement patterns that impact a user's ability to use a computer. FIG. 4 shows a schematic example embodiment of an input filtering system 300 where motion based input to a computer is filtered to remove the distorting effect of abnormal movement. Abnormal movement can negatively impact a person's ability to provide motion based input to a computer by distorting the motion signal. By correcting for a specific abnormal movement pattern an impaired person can accurately and conveniently operate a computer.

User input in the form of motion can be detected by a motion input sensor 302. A motion input sensor 302 can be part of a motion input device. A motion input device can include a traditional computer mouse or any device that converts physical motion by a user into instructions for a computer. In some embodiments the detection hardware can also be configured to provide motion based input to a computer. When operated, a motion input sensor 302 can generate a distorted instruction signal that includes both motion intended by a user as instructions and motion created involuntarily by abnormal movement.

An abnormal movement signal filter 306 can receive a distorted instruction signal 304 from a motion input sensor 302 and produce a corrected instruction signal 308 to a device driver. The abnormal movement signal filter 306 can be software. In some embodiments signal isolation algorithms can be applied by abnormal movement signal filter 306 to detect abnormal movement patterns in a distorted instruction signal 304. The abnormal movement signal filter 306 can utilize identified patterns associated with abnormal movement to identify whether a detected signal should be associated with abnormal movement. Identified abnormal movement patterns can be from a user's specific abnormal movement pattern history, or from a generic identified abnormal movement pattern. A generic identified abnormal movement pattern can be specific to users with certain types or degrees of abnormal movement. Once an abnormal movement pattern is detected in the distorted instruction signal 304, that pattern can be removed from the signal by the abnormal movement signal filter 306. The abnormal movement signal filter 306 can in some embodiments apply Bayesian logic to determine the probability that a detected pattern is associated with abnormal movement. In some embodiments the abnormal movement signal filter 306 can apply other methods of heuristic problem solving to identify abnormal movement patterns in a computer instruction signal.

The corrected instruction signal 308 can be sent to a device driver 310 in the same manner that instructions from the motion input sensor 302 would be. A motion input device including motion input sensor 302 can be associated with a device driver 310. A device driver 310 can be a computer program that operates or controls a particular type of associated device that can be attached to a computer. A device driver 310 can communicate with an associated device through the computer bus or communications subsystem. When a calling program invokes a routine in the device driver 310, the device driver 310 can issue commands to the associated device. Once the associated device sends back data to the device driver 310, the device driver 310 may invoke routines in the original calling program. Data sent back to the device driver 310 from motion input sensor 302 and the associated motion input device can be filtered by abnormal movement signal filter 306.

The apparatus operated by motion based instructions can be a personal computer for individual use. In some embodiments the operated device could be part of a larger system being operated at least in part by a person with abnormal movement such as an interface in an industrial setting or an office computer that is part of a larger network. The abnormal movement signal filter 306 can be housed on or utilize one or more processors. In some embodiments the abnormal movement signal filter can involve one or more processors that are part of the motion input device or part of the computer receiving the instructions. Motion input sensor 302 and device driver 310 can be housed on or utilize one or more processors which can include processors housing or utilized by abnormal movement signal filter 306.

A signal filter can be utilized in some embodiments even where a user is not suffering from a clinical abnormal movement. In some contexts such as laparoscopic surgery a filter identifying and correcting for minimal involuntary muscle movement noise in instruction signals could be useful. The signal corrections in non-abnormal movement settings can in some embodiments be based on previously recorded motion signal input, which can be associated as either a noise input or an intentional input. In some embodiments the same features which could be used to identify a potential abnormal movement if on a larger magnitude can be used to identify instruction signal noise in non-abnormal movement users.

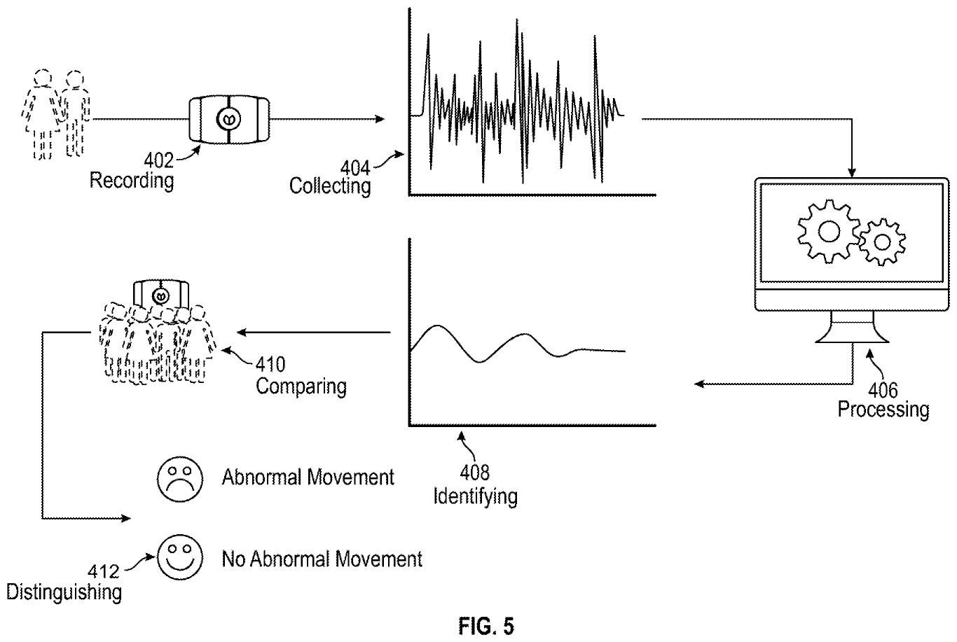

FIG. 5 shows a flow chart of an example embodiment for calibrating methods of detecting abnormal movement. Initially, movements from individual control and abnormal movement patients can be recorded using detection hardware 402. The detection hardware 402 of FIG. 5 can be a device having an accelerometer, an electromyographic sensor, and optionally a gyroscope, in which the device is arranged and designed to be worn on the arm of a human subject. However, the detection of data can be accomplished according to a variety of embodiments, including any embodiment described above in connection with FIG. 1, 2, or 3. Next, movement data and electrical impulse data from muscle activity can be collected 404. Data collection can in some embodiments involve transferring data detected by one or more piece of detection hardware to a data store.

Data can be processed 406 to identify distinct patterns produced by individuals with abnormal movement, in some embodiments by using a signal-noise isolation algorithm. Abnormal movement patterns can then be identified 408. In some embodiments any of the above methods described in connection with FIG. 1, 2, or 3 for identifying an abnormal movement pattern can be used to identify abnormal movement pattern. Data collected from many patients can next be processed and compared 410 against data collected from previously identified abnormal movement patterns. Finally, the system can distinguish 412 between individuals with and without abnormal movement.

During the course of development of the technology herein shown and described, certain experiments were conducted. According to one such experiment, six tremor patients and three non-tremor control subjects were selected for a study. Neither the tremor nor the control subjects had a current or prior history of cognitive impairment, or other neurodegenerative disorder. Tremor detection hardware was attached to each subject. The hardware included: 1) an accelerometer to detect acceleration in Gs, using three channels, 2) a gyroscope to provide rotation rates in degrees/second, using three channels, and 3) an electromyograph providing a set of eight electromyographic sensors that capture electrical activity within a unitless range of −127 to 127 via eight channels, including one for each sensor. In the experiment, the particular detection hardware used was the MYO device developed by Thalmic Labs, Inc. of Kitchener, Ontario, Canada, although any suitable device including the above-referenced criteria could be used.

With the detection hardware attached first on the forearm of the dominant hand, the subjects were asked to carry out certain tasks, in a seated position. The tasks were then repeated with the detection hardware attached to the forearm of the other hand. The first task was to place the hands at rest on thighs to record data at a baseline, during 30-60 seconds. This task assessed any rest tremors. The second task was to stretch the arms straight out, parallel to the floor, with hand palms facing down (to measure postural tremors), and elbows flexed to use the index finger to touch the nose (to measure kinetic tremors), slowly and continuously for 30-60 seconds.

The third task was to, beginning at a single point and expanding outward in a clockwise direction, draw a spiral on a blank sheet of paper using a pen, while avoiding hand contact with the paper. Spiral drawings is a useful instrument to assess amplitude, frequency, and axis of a tremor, and these parameters can in turn indicate a type of tremor. For example, patients with essential tremor are more likely to have a unidirectional tremor axis regardless of position on the spiral. They will also tend to draw the spiral with normal size and spacing. In contrast, patents with dystonic tremor tend to have multiple tremor axes, and patents with Parkinsonian tremor tend to draw small spirals with tight spacing. This task was performed three times with each hand.

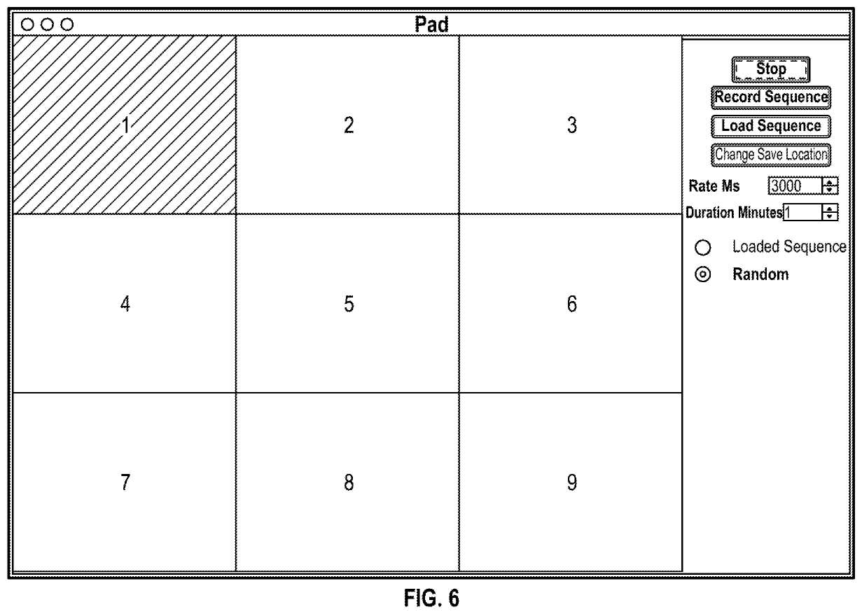

Fourth, the subjects were asked to look at a computer screen displaying a set of nine gray squares arranged in a three-by-three pattern table. FIG. 6 shows a screenshot of the computer screen where square 1 is shaded to represent the color blue while squares 2-9 are gray in color. Software was created to generate a random sequence of color change (e.g., from gray to blue) every three seconds for one square at a time, and the subjects were asked to use a computer mouse to follow, point, and click on each square as it changed color from gray to blue. This task was carried out for 30-60 seconds. Although the screen associated with the fourth task is described in one particular embodiment to include squares that are gray and blue, in practice other shapes than squares can be displayed on the screen, and other colors than gray and blue.

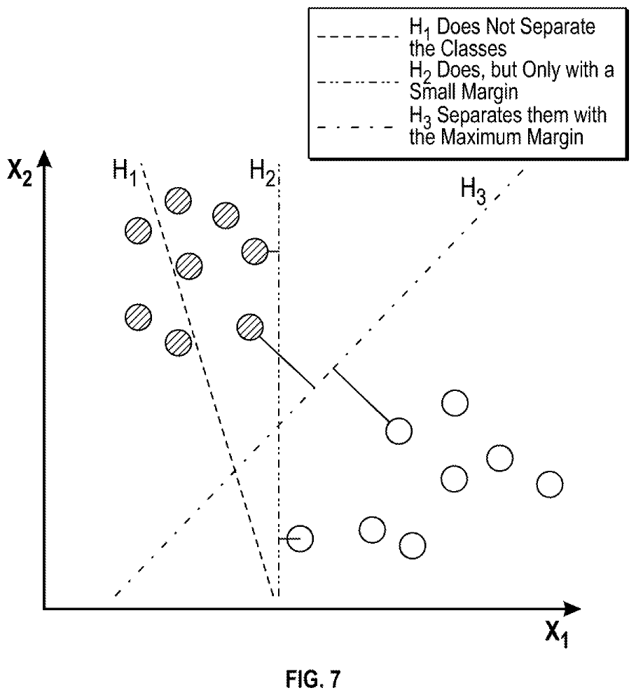

During and after completion of the tasks, data from the detection hardware was segmented into short time intervals during which the signal remained in a consistent frequency range, and the data was converted into a spectrogram for each time interval. Features were extracted from the spectrograms using a toolbox called MatConvNet, and classified with the algorithms support vector machines (SVM) and Random Forest. Support vector machines are a type of machine learning algorithm that, given a set of training data classified into two categories (i.e. tremor and no tremor), maps the data as points in space and attempts to maximize the distance between the two sets of points. New examples are then mapped and predicted to be in the category that corresponds to the side on which the data point falls. FIG. 7 is a graphic showing this concept. Notably, this approach can be useful in image classification and the biological sciences.

In the experiments herein described, frequency vs. time was plotted as an image with the goal of using machine-learning techniques for differentiating images to differentiate between the resulting red-blue-green (RBG) representation. In FIG. 7, H₁ represents a green line, H₂ represents a blue line, and H₃ represents a red line. Red, blue, and green are manifestations of the three primary wavelengths of visible light used to generate color images. Using these three channels is the most common way of storing and reproducing images, as opposed to other color spaces with more specific uses such as cyan-magenta-yellow-key (CMYK), where colors are made from four channels that represent the primary colors of printer ink.

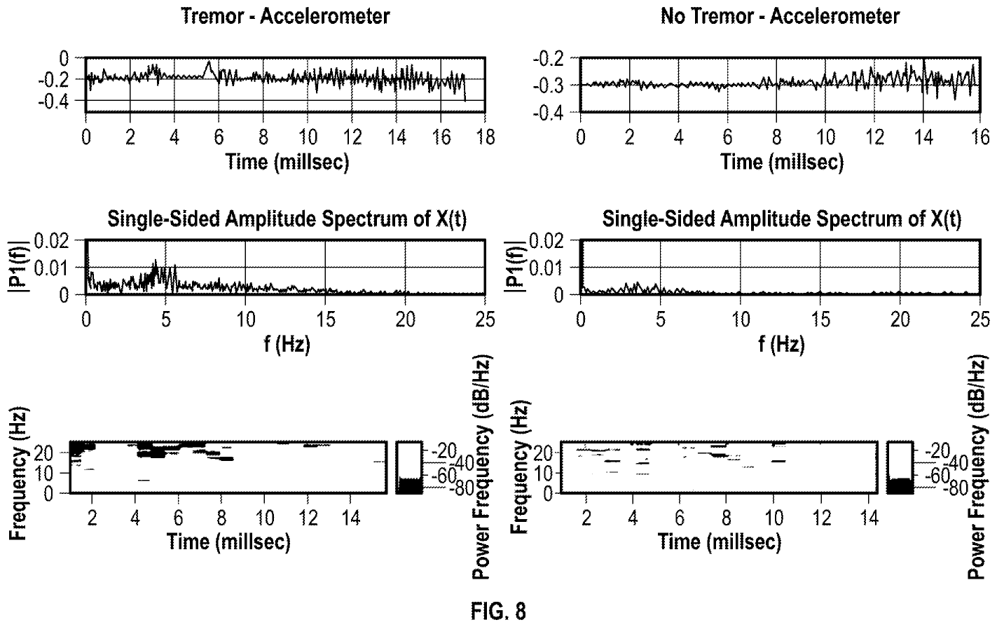

FIGS. 8-10 show plots for the data associated with the accelerometer, gyroscope, and EMG, respectively, in each case collected during task 4 (i.e., the handwriting task). In practical applications, many signals are non-stationary. This means that their frequency-domain representation (their spectrum) changes over time. One can divide almost any time-varying signal into time intervals short enough that the signal is essentially stationary in each section. Time-frequency analysis is most commonly performed by segmenting a signal into those short periods and estimating the spectrum over sliding windows. The spectrogram function computes a Fast Fourier Transform (FFT) based spectral estimate over each sliding window. It allows visualization of how the frequency content of the signal changes over time. Such information is displayed in FIGS. 8-10.

MatConvNet is a toolbox created by the Oxford Vision Geometry Group for implementing convolution neural networks (CNNs) for computer vision applications that contain multiple pre-trained CNNs for tasks such as image classification, segmentation, face recognition, and text extraction. Since tremors vary with individuals, it is helpful to use machine learning algorithms in place of simple discriminator algorithms to identify tremors in different individuals. Some advanced machine learning algorithms operate on images, and can recognize types of objects in images. In the present technology, this approach is used in tremor detection. For example, frequency vs. time data can be plotted as an image (X, Y, Z→RBG). Machine learning techniques can then be used to recognize a picture or representation of a tremor and distinguish it from a picture or representation of a control signal. In the experiments described herein, MatConvNet was used to extract features from the data. This included vgg-face (37 layers, 2622 features) and imagenet-vgg-f (21 layers, 1000 features). FIGS. 8-10 show some example results.

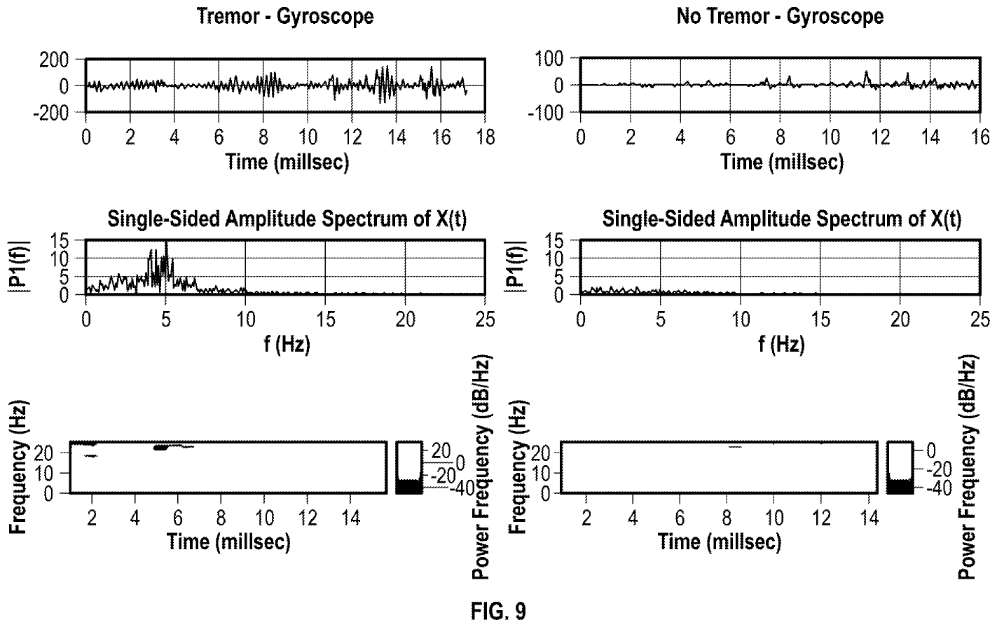

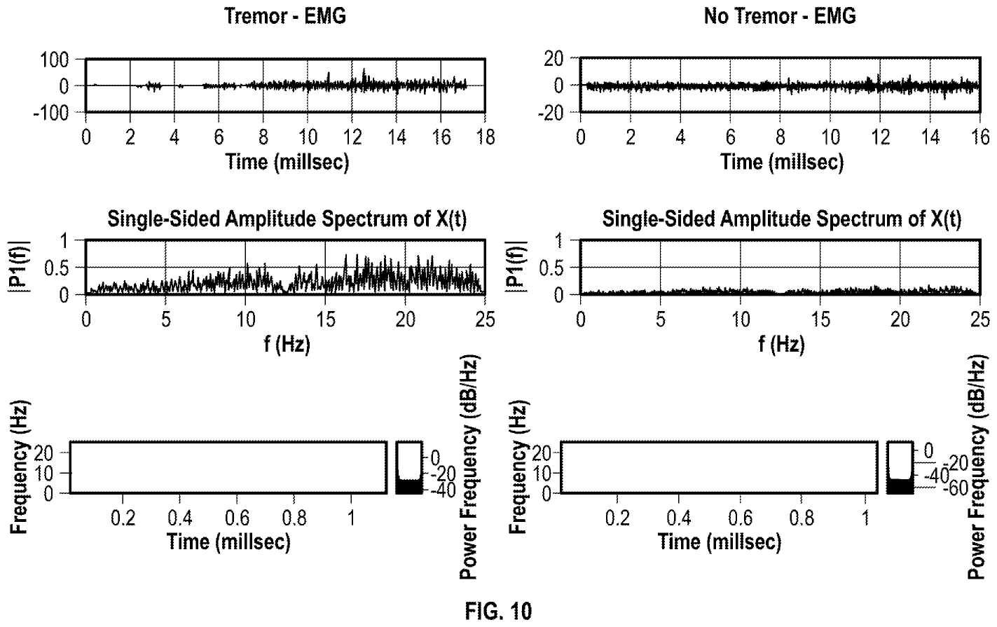

In each of FIGS. 8-10, the top graph plots the collected data, with time represented on the x-axis and units of the specific sensor (i.e., accelerometer in FIG. 8, gyroscope in FIG. 9, and EMG in FIG. 10) represented on the y-axis. The middle graph is a derivative of the top graph, and shows the distribution of amplitude (shown on the y-axis) across the frequency spectrum (shown on the x-axis). As shown in FIGS. 8-10, the data on the middle graph remains in a consistent frequency range. Finally, the bottom graph is a color spectrogram (represented in grayscale), which allows three measurements to be displayed at once, including time, frequency, and amplitude. As shown, time and frequency can be plotted on the x and y axes, respectively, while color or shade represents amplitude or intensity.

Next, all the recorded EMG impulses and kinematic data were processed and formatted for the generation of proof-of-concept algorithms to differentiate signals from control individuals and patients clinically affected with tremors. To do so, the present technology includes a machine learning approach for tremor detection. Specifically, the software can ingest and process the data from both tremor and non-tremor patients which, in some embodiments, may be provided in Excel in a time series format. Thereafter, the software can convert the data to a spectrogram, and extract features from the spectrogram using transfer learning techniques. It is notable that in the spectrums shown in FIG. 8 (Accelerometer) and FIG. 9 (Gyroscope), the tremors show spikes between about 4-6 Hz. In the spectrum shown in FIG. 10 (EMG), there is more energy shown across all bands. Once the data is converted to a spectrogram, the software can then classify features using classification algorithms, and evaluate using, for example, 10 fold cross validation.

Initial testing of the detection hardware relied heavily on the positional and movement data gathered from the accelerometer and gyroscope functions, as this is what has been successfully demonstrated by other researchers, and therefore provided a good starting baseline. One strength, however, of the particular detection hardware used in the study is its inclusion of an 8-lead EMG sensor. Data from this EMG sensor has more noise than the other sensors, and as a result takes longer to optimize, but also allows the software to make comparisons between the actual movements and positional changes that occur (as measured by the accelerometer and gyroscope), and the electrical impulses generated by the muscles of the wearer. This allows software processing the data to distinguish between tremor movements that are occurring when the wearer is at rest, as opposed to tremor movements that are occurring when the wearer is making a purposeful movement. These different types of tremors are known as resting tremors and intention tremors, respectively, and can help neurologists to distinguish between different tremor pathologies to decide on appropriate treatment.

Further benefits of the above described technology, which, as described, includes measurement and analysis of both kinematic and EMG data to measure and analyze tremors, include: 1) provision of data to software that can compensate for abnormal tremors to allow impaired people to operate a computer where they otherwise could not, 2) provision of allocation criteria in clinical trials, 3) reduction of the need to visit a health provider regularly, as data measurement can occur remotely without clinician help, 4) provision of data related to the response of tremors to medication, and 5) provision of earlier diagnosis of certain disorders, such as Parkinson's or Alzheimer's disease, by providing a means to differentiate asymptomatic tremors from those that might develop into such diseases.

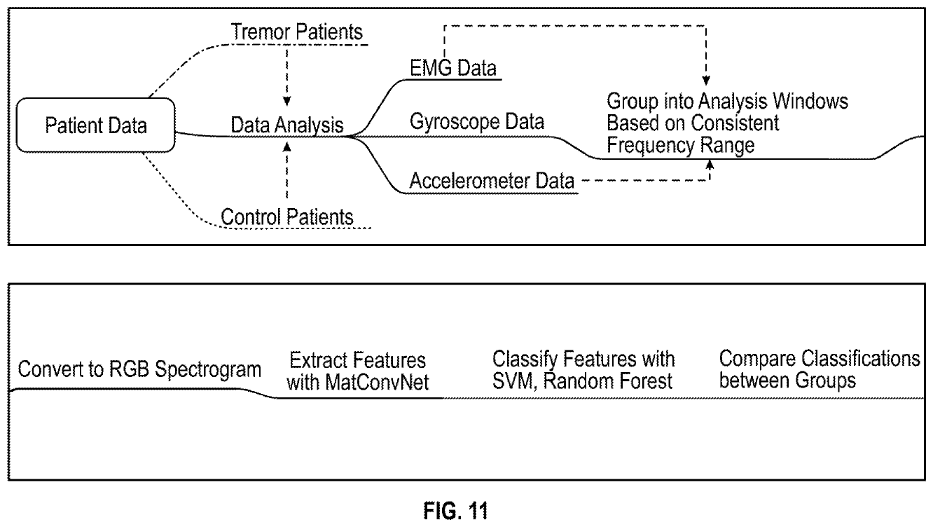

FIG. 11 shows a schematic example of an embodiment of an experiment consistent with the above described example experiments. As shown, two sets of patient data can be initially provided, including control or baseline data and data collected from the tremor patients during the experiments. All of the data can consist of accelerometer data, gyroscope data, and EMG data. The data can be grouped into analysis windows based on a demonstrated consistent frequency range (e.g., as shown in the plot diagrams of FIGS. 8-10. Thereafter, the data can be converted to an RGB spectrogram, also similar to those shown in FIGS. 8-10. Specific features of the movements or tremors of each patient can then be extracted using, for example, MatConvNet, and classified using, for example, SVM or Random Forest, as described above. Classifications between groups can be compared to help analyze different types of movement and identify specific types of tremors.

The systems and methods disclosed herein can be implemented in hardware, software, firmware, or a combination thereof. Software can include computer-readable instructions stored in memory (e.g., non-transitory, tangible memory, such as solid state memory (e.g., ROM, EEPROM, FLASH, RAM), optical memory (e.g., a CD, DVD, Blu-ray disc, etc.), magnetic memory (e.g., a hard disc drive), etc.), configured to implement the algorithms on a general purpose computer, special purpose processors, or combinations thereof. For example, one or more computing devices, such as a processor, may execute program instructions stored in computer readable memory to carry out processes disclosed herein. Hardware may include state machines, one or more general purpose computers, and/or one or more special purpose processors. In some embodiment, multiple processors can be used, and in some implementations the processors can be at different locations (e.g., coupled via a network). While certain types of user interfaces and controls are described herein for illustrative purposes, other types of user interfaces and controls may be used.

The embodiments discussed herein are provided by way of example, and various modifications can be made to the embodiments described herein. Certain features that are described in this disclosure in the context of separate embodiments can also be implemented in combination in a single embodiment. Conversely, various features that are described in the context of a single embodiment can be implemented in multiple embodiments separately or in various suitable subcombinations. Also, features described in connection with one combination can be excised from that combination and can be combined with other features in various combinations and subcombinations. Various features can be added to the example embodiments disclosed herein. Also, various features can be omitted from the example embodiments disclosed herein.

This application is a PCT application, and claims priority to, and the benefit of, U.S. Provisional Application No. 62/634,474, filed Feb. 23, 2018, titled “SYSTEMS AND METHODS FOR DETECTION AND CORRECTION OF ABNORMAL MOVEMENTS,” which is incorporated herein by reference in its entirety.

Similarly, while operations are depicted in the drawings or described in a particular order, the operations can be performed in a different order than shown or described. Other operations not depicted can be incorporated before, after, or simultaneously with the operations shown or described. In certain circumstances, parallel processing or multitasking can be used. Also, in some cases, the operations shown or discussed can be omitted or recombined to form various combinations and sub combinations.

What is claimed is:

1. A device to detect tremors in human subjects, the device comprising:
an accelerometer to measure muscle movement in the human subject and generate muscle movement data; and
an electromyographic sensor to measure electrical impulses generated by muscles of the human subject and generate electrical impulse data, the muscle movement data and electrical impulse data being capable of comparison to predetermined baseline data to identify abnormal movements of the human subject.

2. The device of claim 1, further comprising:
a gyroscope to measure muscle movement in the human subject and generate muscle movement data.

3. The device of claim 1, wherein the device is arranged and designed to be worn on the arm of the human subject.

4. The device of claim 1, wherein the accelerometer measures three-dimensional muscle movement.

5. The device of claim 4, wherein the device is capable of communicating muscle movement and electrical impulse data to a computer via wired connection, a wireless network, a Bluetooth connection, or a cellular phone link, and wherein the electromyographic sensor detects electrical impulses via electrode leads when attached to the human subject.

6. (canceled)

7. A method for measuring tremor activity in a human subject, the method comprising:
a) detecting muscle movement in the human subject with a kinetic sensor;
b) detecting electrical impulses from muscles in the human subject with an electromyographic sensor;
c) comparing data associated with the muscle movement and the electrical impulses with predetermined baseline data to identify abnormalities in the data; and
d) analyzing the abnormalities in the data associated with the muscle movement and the electrical impulses to identify abnormal movement in the human subject.

8. The method of claim 7, wherein step c) further comprises:
detecting electrical impulses from muscles of the human subject or other human subjects before muscle movement;
predicting muscle activity and associated electrical impulses based on pre-movement electrical impulses, such predicted muscle activity and associated electrical impulses comprising the baseline data.

9. The method of claim 7, wherein step c) further comprises:
collecting known muscle movement and electrical impulse data based on properties of the detected movement;
predicting muscle activity and associated electrical impulses based on the collected known muscle movement and electrical impulse data, such predicted muscle activity and associated electrical impulses comprising the baseline; and
identifying deviations or aberrations in the detected muscle movement and electrical impulses relative to the predetermined baseline data.

10. (canceled)

11. The method of claim 7, wherein step d) further comprises:
applying an algorithm associated with a known disorder to the detected muscle movement and electrical impulse data to verify the existence of the known disorder in the human subject.

12. The method of claim 11, wherein the known disorder is selected from the group consisting of tremors, including tremors associated with Parkinson's or Alzheimer's disease, myoclonus, chorea, athetosis, balismus, and bradykinesia.

13. The method of claim 7, wherein the kinetic sensor is an accelerometer or a gyroscope, or both and accelerometer and a gyroscope.

14. A computer-implemented method comprising:
under the control of one or more computer systems configured with executable instructions,
a) detecting both intended motion and involuntary motion of a user with an input sensor;
b) generating a distorted instruction signal including data related to both the intended motion and the involuntary motion;
c) sending the distorted instruction signal to a signal filter;
d) producing a corrected instruction signal stripped of the involuntary motion; and
e) sending the corrected instruction signal to a device driver.

15. The method of claim 14, wherein the input sensor is part of a motion input device, and the motion input device converts physical motion by the user into instructions for a computer.

16. The method of claim 15, wherein the motion input device is a computer mouse, and wherein the signal filter is computer software.

17. (canceled)

18. The method of claim 14, wherein step d) further comprises:
identifying patterns associated with abnormal movement;
comparing the patterns associated with abnormal movement with the distorted instruction signal to isolate portions of the distorted instruction signal associated with abnormal movement; and
separating the portions of the distorted instruction signal associated with abnormal movement from the rest of the signal to generate the corrected instruction signal; and
applying Bayesian logic to determine the probability that a detected pattern associated with abnormal movement is associated with abnormal movement, the device driver being a computer program that controls an associated device.

19. (canceled)

20. (canceled)
